(19) United States
(12) Patent Application Publication
(10) Pub. No.: US 2025/0025386 A1
(43) Pub. Date: Jan. 23, 2025

(54) Engineered liposome with cell membrane proteins to reduce melanosome transport

(71) Applicants: Jiangnan University, Wuxi (CN); Harvard University, Cambridge, MA (US)

(72) Inventors: Cheng Yang, Wuxi (CN); Chunhuan Liu, Wuxi (CN); Yuchun Liu, Wuxi (CN); Kevin Jahnke, Cambridge, MA (US); David A. Weitz, Cambridge, MA (US)

(21) Appl. No.: 18/907,682

(22) Filed: Oct. 7, 2024

Related U.S. Application Data
(63) Continuation of application No. PCT/CN2023/131260, filed on Nov. 13, 2023.

(30) Foreign Application Priority Data
Nov. 13, 2023 (CN) 202311504100.3

(51) Int. Cl. A61K 8/14; A61K 8/64; A61Q 19/02

(52) U.S. Cl. CPC A61K 8/14; A61K 8/64; A61Q 19/02

(57) ABSTRACT

The present disclosure discloses an engineered liposome with cell membrane proteins to reduce melanosome transport and a preparation method thereof, and belongs to the technical field of cosmetics and biomedicine. The present disclosure provides the engineered liposome with cell membrane proteins to reduce melanosome transport and the preparation method thereof, which is easy to operate, requires no large-scale equipment, has few additives, and a preparation process is simple and environmentally friendly. The biomimetic liposome can significantly inhibit melanin transport. The fluorescence intensity of melanosomes in keratinocytes is found to decrease by 3.5-fold in a co-culture test of melanocytes and the keratinocytes, indicating that this biomimetic liposome is very effective in inhibiting accumulation of melanin in skin keratinocytes. These findings provide an effective strategy for reducing melanosome transfer to treat hyperpigmentation, while also introducing an alternate approach for regulating cellular communication of extracellular vesicles and organelles.

### TECHNICAL FIELD

The present disclosure relates to an engineered liposome with cell membrane proteins to reduce melanosome transport, and belongs to the fields of cosmetics and nanomedicine technologies.

### BACKGROUND

Hyperpigmentation causes certain areas of the skin to become darker than surrounding skin. Generally, hyperpigmentation includes chloasma, senile plaque, and post-inflammatory pigmentation, which can severely affect the patients' appearance and mental health. Hyperpigmentation is caused by an increase in melanin in keratinocytes. While many therapeutic methods focus on inhibiting melanin production through chemotherapy or physical therapy, there is no effective treatment strategy for hyperpigmentation. Skin pigmentation basically results from accumulation of melanin granules in keratinocytes, and this process involves melanin production as well as melanosome transport. Melanosome transport plays a key role in pigmentation of mammalians, and this process is driven by intercellular transfer of melanosomes from melanocytes to keratinocytes. Dysregulation of the melanosome transfer process results from unregulated melanin formation in melanocytes, which in turn causes hyperpigmentation. There are two main mechanisms of melanosome transfer. In one mechanism, the melanosomes are produced from melanocytes. The melanosomes sprout from the apical and central regions in the dendrites of melanocytes via a shedding vesicle mechanism, are released into an extracellular space to form pigmented spheres, and are then phagocytosed by keratinocytes. In the other mechanism, the melanosomes are not transferred through releasing into the extracellular space, but are released and taken up after direct contact of the melanocytes with the keratinocytes. The lateral or apical regions in the dendrites of the melanocytes adhere to the surface of the keratinocytes, and then the dendrites of the melanocytes at the point of contact thin out and shed by themselves to form the melanosomes, which are ultimately taken up by the keratinocytes through phagocytosis. Most importantly, these two transfer mechanisms suggest that melanosome transfer involves recognition of keratinocyte membranes. Most of the existing whitening products are dedicated to inhibiting melanogenesis, which is often less than satisfactory, and whitening ingredients such as Rhododendrol and hydroquinone can cause significant side effects. Inhibiting melanin transport is another way to achieve a whitening effect. If the melanin that has been produced cannot be transported between cells, it will not cause visual darkening. However, it is difficult to treat skin pigmentation through the inhibition of melanosome transport, and the main challenges are the complexity of the melanosome transport mechanism and target specificity. The melanosome transport involves complex interactions among multiple proteins and the cytoskeleton, such as Rab27a and its effector protein MyoVa, as well as dynamic regulation of the microtubule and actin cytoskeleton. Since these proteins and cytoskeletal structures also play key roles in many other cellular processes, non-specific inhibition may lead to a wide range of abnormal cellular functions and side effects. Effective inhibition of this process requires the development of highly specific molecules or drugs, and the drugs should precisely target the transport mechanism without interfering with other key cellular functions to avoid potential side effects. This high requirement of complex transport mechanism and the target specificity make it a great challenge to apply this strategy in the clinic.

### SUMMARY

In view of the problems as mentioned above and/or in the prior art, the present disclosure is proposed.

The present disclosure is based on the fact that keratinocytes are recipient cells for melanin secretion by melanocytes, and therefore, keratinocyte membrane proteins are embedded into a phospholipid bilayer to mimic keratinocytes using microfluidics. This biomimetic strategy can maintain the biological characteristics and functions of the keratinocytes inherent to their role as the recipient cells for melanin transport. Utilizing these biological characteristics and functions, this engineered liposome with cell membrane proteins (biomimetic liposome engineered with membrane proteins from keratinocytes) can be involved in the melanin transport process in skin. Firstly, due to the presence of receptors and ligands on the membrane surface, biomimetic liposome engineered with membrane proteins from keratinocytes will anchor to surfaces of pigmented spheres, which would affect the uptake of these liposomes by keratinocytes as the pigmented spheres begin to transport the melanin to the keratinocytes. Based on the cellular uptake results, the uptake of the biomimetic liposome engineered with membrane proteins from keratinocytes by the keratinocytes will be significantly reduced, and therefore the keratinocytes will reduce the uptake of the pigmented spheres recognized as the biomimetic liposome engineered with membrane proteins from keratinocytes, which will inhibit melanosome transport.

The present disclosure provides an engineered liposome with cell membrane proteins, i.e., a biomimetic liposome engineered with membrane proteins from keratinocytes. The biomimetic liposome includes keratinocyte membrane proteins and lipids with a solution of lipids in ethanol as an internal phase and a solution of the keratinocyte membrane proteins in PBS buffer as an external phase.

In one embodiment, the phospholipid bilayer includes soy lecithin and cholesterol, and the molar ratio of the soy lecithin to the cholesterol is (3-2):1.

In one embodiment, the mass ratio of the keratinocyte membrane proteins to total lipids is 1:(50-500).

In one embodiment, the keratinocytes include, but are not limited to, human immortalized keratinocytes.

In one embodiment, the human keratinocyte membrane proteins are prepared by the following steps. About 20 million to 40 million human immortalized keratinocytes are cultured, the cells are digested with a cell dissociation solution containing EDTA but no trypsin, centrifuged, collected, and washed with PBS pre-cooled in an ice bath. 1 ml of membrane protein extraction reagent A, to which PMSF is added before use, is added to approximately 40 million human immortalized keratinocytes using a cell membrane protein and plasma protein extraction kit (Beyotime), and the cells are fully suspended and then placed in an ice bath for 10 min. The cell suspension is transferred to a 2 ml glass homogenizer pre-cooled in an ice bath, homogenized until the cells are sufficiently fragmented, and then centrifuged at 4° C. and 700 g for 10 min and the supernatant is collected. Centrifugation is performed at 4° C. and 14000 rpm for 30 min, and precipitates are collected. 400 μl of membrane protein extraction reagent B is added, vortexed for 5 s and then placed in an ice bath for 10 min. The process is repeated three times. Subsequently, centrifugation is performed at 4° C. and 14000 g for 5 min, and the supernatant is collected as a membrane protein solution, which is stored at −80° C.

In one embodiment, the membrane protein solution is measured using a BCA protein concentration assay kit (Beyotime).

The present disclosure also provides a method for preparing an engineered liposome with cell membrane proteins.

In one embodiment, the method includes the following steps.

Extraction of human keratinocyte membrane protein: a membrane protein solution is extracted from cultured human immortalized keratinocytes according to a protocol of the cell membrane protein and plasma protein extraction kit (Beyotime) and stored at −80° C.

Preparation of biomimetic liposome engineered with membrane proteins from keratinocytes: soy lecithin and cholesterol are dissolved in anhydrous ethanol to form an organic phase; and the membrane protein solution is diluted with 1×PBS to form an aqueous phase. The organic phase is used as an internal phase and the aqueous phase is used as an external phase. A microfluidic chip is focused via fluid at a certain TFR and a certain FRR. The collected solution is dialyzed overnight at 4° C. to obtain the biomimetic liposome engineered with membrane proteins from keratinocytes, which is stored at 4° C.

As a preferred embodiment of the present disclosure, where: as for that the microfluidic chip is focused via fluid, TFR=100 to 1000 μL/min and FRR=3:1 to 6:1.

As a preferred embodiment of the present disclosure, where: as for the dialysis overnight, a dialysis bag has a molecular weight cut-off of 30 to 300 kDa.

In one embodiment, the molar ratio of soy lecithin to cholesterol in step (1) is (3-2):1; and the concentration of the organic phase is 5 to 10 mg/ml.

The present disclosure also provides a method for inhibiting melanin transfer in skin using the biomimetic liposome or treating skin hyperpigmentation using the biomimetic liposome, where the biomimetic liposome or a product containing the biomimetic liposome is used as a cosmetic, a skincare product, or a medicine.

In one embodiment, the medicine includes a topically applied medicine or an orally administered medicine.

According to the present disclosure, the human keratinocyte membrane proteins are embedded into a liposomal phospholipid bilayer to form a biomimetic liposome using microfluidics, thereby constructing a biomimetic targeting nanoparticle for inhibiting melanin transfer in skin for the treatment of skin hyperpigmentation. According to the present disclosure, an engineered liposome with cell membrane proteins is constructed. The biomimetic liposome is delivered into viable epidermis through a microneedle, and can effectively stay in the viable epidermis layer due to the existence of intercellular adhesion protein, and then can significantly increase the drug retention in the viable epidermis. At the same time, pigmented spheres will be surrounded by a circle of biomimetic liposome engineered with membrane proteins from keratinocytes based on the inherent biological characteristics of keratinocytes. When the pigmented spheres come into contact with the keratinocytes, the recognition and phagocytosis of the pigmented spheres by the keratinocytes will be changed, allowing the carrier itself to play a role of inhibiting melanosome transport. If cosmetic actives such as vitamin C, arbutin, and niacinamide, which treat skin pigmentation through different mechanisms, are encapsulated, a synergistic effect will be achieved.

### Beneficial Effects

The present disclosure provides the engineered liposomes with cell membrane proteins to reduce melanosome transport and the preparation method thereof, which is easy to operate, requires no large-scale equipment, has few additives, and a preparation process is simple and environmentally friendly.

According to the present disclosure, a microfluidic chip is focused via fluid, and self-assembly of phospholipid and membrane proteins occurs within a chip channel, resulting in a membrane protein biomimetic liposome with a particle size of 50-100 nm and a polydispersity index of less than 0.2, as well as low cytotoxicity and good biocompatibility. In co-culture of the keratinocytes and the melanocytes, the biomimetic liposome engineered with membrane proteins from keratinocytes prepared by the present disclosure can inhibit melanin transport to keratinocytes, resulting in a 3.5-fold decrease in melanin transport amount.

The membrane proteins in the present disclosure are membrane proteins of the human immortalized keratinocytes, which are embedded in a liposomal phospholipid bilayer to form a biomimetic nanoliposome by focusing a microfluidic chip via fluid. Keratinocytes are recipient cells for melanin transported by melanocytes, and their cell membranes are equipped with a series of complex surface receptors that enable them to respond to biosignals of the melanocytes. Therefore, the biomimetic liposome engineered with membrane proteins from keratinocytes can effectively adhere around the loaded pigmented spheres and alter the recognition of pigmented spheres by the keratinocytes, thereby reducing the number of melanosomes entering the keratinocytes. In addition, the presence of adhesion proteins on cell surface increases the retention of the biomimetic liposome engineered with membrane proteins from keratinocytes in the viable epidermis, thereby substantially increasing the bioavailability of the biomimetic liposome engineered with membrane proteins from keratinocytes and decreasing the toxicity in vivo.

The keratinocyte membrane protein liposome prepared by the present disclosure has a melanosome transport blocking effect in the carrier itself, and if combined with the encapsulated melanogenesis inhibitor, melanin transport inhibitor and the like, it can have synergistic effects, thus increasing the therapeutic effect on skin pigmentation.

### BRIEF DESCRIPTION OF FIGURES

FIG. 1 is a structural diagram of a PDMS microfluidic chip.

FIG. 2A is a diagram showing particle sizes of biomimetic liposome engineered with membrane proteins from keratinocytes according to the present disclosure.

FIG. 2B is a diagram showing polydispersity indexes of biomimetic liposome engineered with membrane proteins from keratinocytes according to the present disclosure.

FIG. 2C is a diagram showing a Zeta potentials of biomimetic liposome engineered with membrane proteins from keratinocytes according to the present disclosure.

FIG. 3 is a polyacrylamide gel electropherogram (SDS-PAGE) of human immortalized keratinocyte membrane proteins and biomimetic liposome engineered with membrane proteins from keratinocytes extracted in the present disclosure.

FIG. 4 is an SEM diagram of biomimetic liposome engineered with membrane proteins from keratinocytes according to the present disclosure.

FIG. 5 is a Cryo-TEM diagram of a biomimetic liposome engineered with membrane proteins from keratinocytes according to the present disclosure.

FIG. 6A is a HaCaT cytotoxicity diagram of a biomimetic liposome engineered with membrane proteins from keratinocytes according to the present disclosure.

FIG. 6B is an MNT-1 cytotoxicity diagram of a biomimetic liposome engineered with membrane proteins from keratinocytes according to the present disclosure.

FIG. 6C is a diagram showing cytotoxicity of a co-culture model of biomimetic liposome engineered with membrane proteins from keratinocytes according to the present disclosure.

FIG. 7 is a diagram showing flow analysis for cellular uptake of biomimetic liposome engineered with membrane proteins from keratinocytes according to the present disclosure.

FIG. 8A is a diagram showing results of flow cytometry analysis of biomimetic liposome engineered with membrane proteins from keratinocytes for inhibition of melanosome transport in a co-culture model according to the present disclosure.

FIG. 8B is a graph showing PMEL 17 fluorescence intensity within a Q2 region in flow cytometry analysis of biomimetic liposome engineered with membrane proteins from keratinocytes for inhibition of melanosome transport in a co-culture model according to the present disclosure.

FIG. 9 is a diagram showing the number of PMEL 17 fluorescent bright spots within an individual keratinocyte in a laser confocal analysis of biomimetic liposome engineered with membrane proteins from keratinocytes for inhibition of melanosome transport in a co-culture model according to the present disclosure.

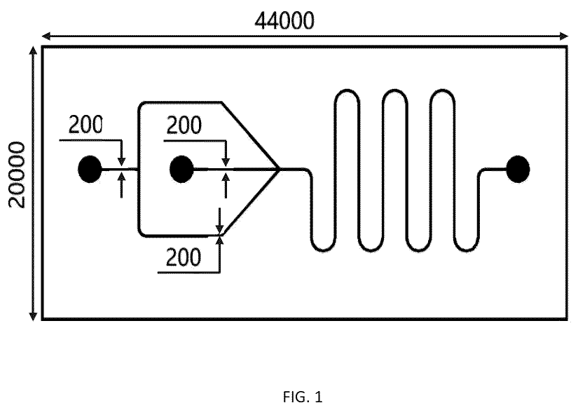

### DETAILED DESCRIPTION

In order to make the above objectives, features and advantages of the present disclosure more obvious and easy to understand, the following specific embodiments of the present disclosure are described in detail in conjunction with examples of the specification.

Many specific details are set forth in the following description in order to facilitate a full understanding of the present disclosure. However, the present disclosure can be implemented in other ways different from those described herein, and a person skilled in the art can make similar generalizations without violating the concepts of the present disclosure, and thus the present disclosure is not limited by the specific examples disclosed below.

In addition, “an example” or “example” as used herein refers to a particular feature, structure, or characteristic that may be included in at least one embodiment of the present disclosure. The expression “in one example” appearing in different places in this specification do not all refer to the same example, nor are they separate examples or examples selectively mutually exclusive with other examples.

### Examples 1-3: Extraction of Human Immortalized Keratinocyte Membrane Proteins

Well-grown human immortalized keratinocytes HaCaT were taken, and the cells were digested with a cell dissociation solution containing EDTA but no trypsin, centrifuged, collected, and washed with PBS pre-cooled in an ice bath. A membrane protein extraction reagent A, to which phenylmethylsulfonyl fluoride was added before use, was added to approximately 40 million human immortalized keratinocytes using a cell membrane protein and plasma protein extraction kit (Beyotime), and the cells were fully suspended and then placed in an ice bath for 10 min. The cell suspension was transferred to a glass homogenizer pre-cooled in an ice bath, homogenized until the cells were sufficiently fragmented, and then centrifuged at 4° C. and 700 g for 10 min and the supernatant was collected. Centrifugation was performed at 4° C. and 14000 rpm for 30 min, and precipitates were collected. A membrane protein extraction reagent B was added, vortexed for 5 s and then placed in an ice bath for 10 min. The process was repeated three times. Subsequently, centrifugation was performed at 4° C. and 14000 g for 5 min, and the supernatant was collected as a membrane protein solution, which was stored at −80° C.

The extraction processes for the human immortalized keratinocyte membrane proteins in the Examples are shown in Table 1:

TABLE 1

| Process Condition | Example 1 | Example 2 | Example 3 |
|---|---|---|---|
| Number of | 20 | 30 | 40 |
| Homogenization | | | |
| Membrane Protein | 0.3 | 0.3 | 0.3 |
| Extraction Reagent B | | | |
| (ml) | | | |
| Membrane Protein | 0.67 | 1.15 | 1.16 |
| Concentration | | | |
| (mg/ml) | | | |

### Examples 4-13: Preparation of Biomimetic Liposome Engineered with Membrane Proteins from Keratinocytes

A certain amount of soy lecithin and cholesterol were mixed and dissolved in anhydrous ethanol at a total lipid concentration of 10 mg/ml to be prepared as an organic phase. A certain amount of keratinocyte membrane protein solution (prepared in Example 2) was dissolved in 1×PBS buffer to be prepared as an aqueous phase, with a ratio of the mass concentration of membrane proteins to the mass concentration of total lipids of 1:(100-300). The aqueous phase was used as an external phase and the organic phase was used as an internal phase. A microfluidic chip (Wuhan Jianmizhikong Technology Co., Ltd.) was focused via fluid at a certain total flow rate (TFR) and a certain flow rate ratio (volume flow rate of aqueous phase/volume flow rate of organic phase=FRR). An obtained solution was dialyzed overnight with a 300 kDa dialysis bag to obtain the biomimetic liposome engineered with membrane proteins from keratinocytes.

From Examples 4-6, it can be concluded that as the ratio of the soy lecithin to the cholesterol was increased, the particle size of the prepared liposomes showed a downward and then upward trend, which were 181.4 nm, 120.3 nm, and 130.1 nm, respectively, and the polydispersity index was decreased, which were 0.192, 0.179, and 0.181, respectively. From Examples 5, 7, and 8, it can be concluded that as the TFR was increased, the particle size of the prepared liposomes was decreased, which were 120.3 nm, 97.6 nm, and 57.2 nm, respectively, and the polydispersity index was slightly increased, which were 0.179, 0.184, and 0.197, respectively. Example 8 was preferably selected as the preferred process for membrane protein embedding based on indicators of smaller particle size and lower polydispersity index.

TABLE 2
Process conditions for preparation of biomimetic liposome engineered
with membrane proteins from keratinocytes in Examples 4-13

| Process Condition | 4 | 5 | 6 | 7 | 8 | 9 | 10 | 11 | 12 | 13 |
|---|---|---|---|---|---|---|---|---|---|---|
| Soy | 6:3 | 7:3 | 8:3 | 7:3 | 7:3 | 7:3 | 7:3 | 7:3 | 7:3 | 7:3 |
| lecithin:cholesterol | | | | | | | | | | |
| (molar ratio) | | | | | | | | | | |
| TFR (μL/min) | 500 | 500 | 500 | 700 | 1000 | 1000 | 1000 | 1000 | 1000 | 1000 |
| FRR | 6:1 | 6:1 | 6:1 | 6:1 | 6:1 | 6:1 | 6:1 | 6:1 | 6:1 | 6:1 |
| Membrane Protein | 0 | 0 | 0 | 0 | 0 | 0.1 (1:100) | 0.05 (1:200) | 0.033 (1:300) | 0.05 | 0 |
| Concentration (mg/ml) | | | | | | | | | | |
| Rhodamine B-DHPE | 0 | 0 | 0 | 0 | 0 | 0 | 0 | 0 | 0.2 | 0.2 |
| Concentration (mol %) | | | | | | | | | | |

The properties of the engineered liposomes with cell membrane proteins were characterized in the present disclosure:

1. Characterization of Particle Size and Potential of the Biomimetic Liposome Engineered with Membrane Proteins from Keratinocytes

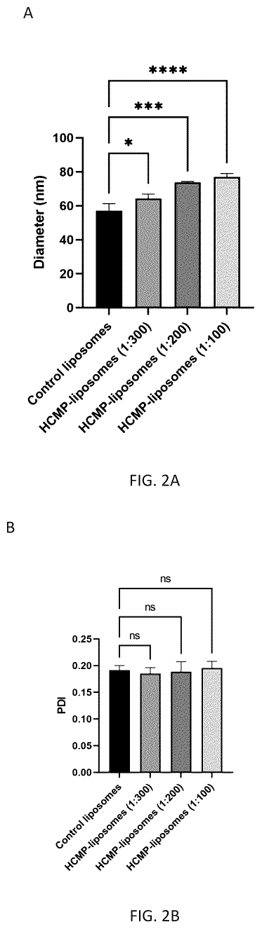

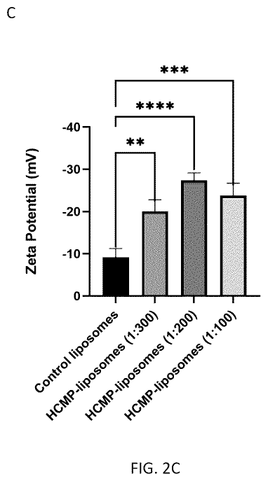

The particle size, polydispersity index and Zeta potential of liposomes without membrane proteins (Liposomes) (prepared in Example 8) and biomimetic liposome engineered with membrane proteins from keratinocytes with different concentrations of membrane proteins (HCMP-liposomes) (prepared in Examples 9-11) were measured by using a zeta potential and nanoparticle size analyzer. The results are shown in FIG. 2. It can be seen that with the incorporation of membrane proteins and the increase of membrane protein concentration, the average particle size of the prepared biomimetic liposome engineered with membrane proteins from keratinocytes was increased, the Zeta potential was decreased and then increased, and the polydispersity index did not change significantly, which indirectly indicated that the membrane proteins were successfully embedded. While the Zeta potential of the biomimetic liposomes should be decreased with the increased embed of membrane proteins, the Zeta potential of the biomimetic liposome engineered with membrane proteins from keratinocytes with the membrane protein concentration of 0.1 mg/ml (prepared in Example 9) was increased compared with that of the biomimetic liposome engineered with membrane proteins from keratinocytes with the membrane protein concentration of 0.05 mg/ml (prepared in Example 10), indicating that the membrane protein concentration of 0.1 mg/ml was oversaturated. In order to avoid wasting the membrane proteins, the biomimetic liposome engineered with membrane proteins from keratinocytes with the membrane protein concentration of 0.05 mg/ml (prepared in Example 10) was preferred, of which the particle size was 73.83 nm and the polydispersity index was 0.189.

2. Characterization of Polyacrylamide Gel Electrophoresis (SDS-PAGE) of the Biomimetic Liposome Engineered with Membrane Proteins from Keratinocytes

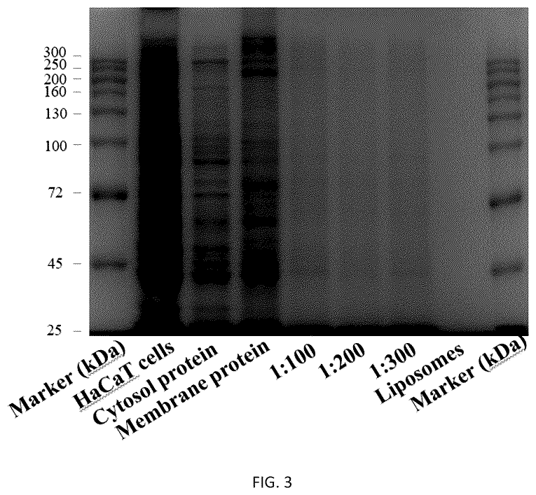

The whole protein, plasma protein, and membrane protein of human immortalized keratinocytes, and biomimetic liposome engineered with membrane proteins from keratinocytes with different membrane protein concentrations (prepared in Examples 8-11) were detected by SDS-PAGE. The human immortalized keratinocytes and the biomimetic liposome engineered with membrane proteins from keratinocytes with different membrane protein concentrations were first lysed with a cell lysate, quantified with BCA proteins and then subjected to gel electrophoresis. The results showed that the membrane protein band and the plasma protein band were significantly different for the human immortalized keratinocytes, and the superposition of the two was similar to the whole protein of the human immortalized keratinocytes, indicating that the extraction of the human immortalized keratinocyte membrane proteins was successful. The protein bands of the biomimetic liposome engineered with membrane proteins from keratinocytes with different membrane protein concentrations were basically the same as that of the membrane proteins, indicating that the membrane proteins were successfully embedded. The results are shown in FIG. 3.

3. Characterization of Scanning Electron Microscope (SEM) of the Biomimetic Liposome Engineered with Membrane Proteins from Keratinocytes

The microscopic morphology of biomimetic liposome engineered with membrane proteins from keratinocytes (prepared in Example 10), and liposomes without membrane proteins (prepared in Example 8) were observed by a field emission scanning electron microscope for characterization. A certain amount of trehalose was added to the liposome preparation as a lyoprotectant and formulated into a trehalose liposome solution with a final concentration of 100 mM, which was snap-frozen with liquid nitrogen and then placed in a lyophilizer for lyophilization. After completion of lyophilization, the lyophilized samples were coated onto a conductive adhesive and placed under a scanning electron microscope for observation.

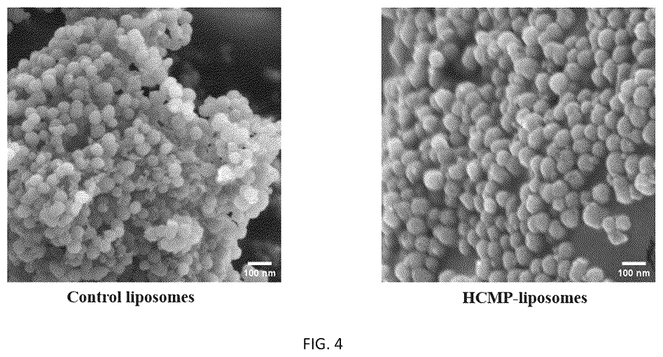

The results are shown in FIG. 4. It can be seen that the biomimetic liposome engineered with membrane proteins from keratinocytes prepared by microfluidics and liposomes without membrane proteins were spherical and had well-defined edges, although their sizes differed from those measured by a zeta potential and nanoparticle size analyzer, which might be due to aggregation occurring caused by structural disruption of the liposomes during the process of snap-freeze with liquid nitrogen as well as during the process of lyophilization.

4. Characterization of Cryo-Transmission Electron Microscope (Cryo-TEM) of the Biomimetic Liposome Engineered with Membrane Proteins from Keratinocytes

The microscopic morphology of biomimetic liposome engineered with membrane proteins from keratinocytes (prepared in Example 10), and liposomes without membrane proteins (prepared in Example 8) were observed by a cryo-transmission electron microscope for characterization.

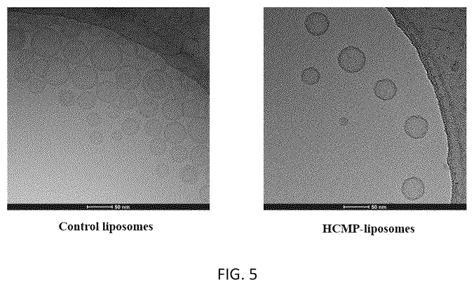

The results are shown in FIG. 5. It can be seen that the biomimetic liposome engineered with membrane proteins from keratinocytes were spherical and were all unilamellar vesicles, and the liposomes without membrane proteins were of slightly irregular spheres and showed a small number of multilamellar vesicles. The sizes all matched the particle size data measured by the zeta potential and nanoparticle size analyzer.

5. Investigation of Cytotoxicity of the Biomimetic Liposome Engineered with Membrane Proteins from Keratinocytes

The cytotoxicity of the biomimetic liposome engineered with membrane proteins from keratinocytes and protein-free liposomes on human immortalized keratinocytes (HaCaT cells), human melanoma cells (MNT-1 cells), and a co-culture model of the two cells (the ratio of HaCaT to MNT-1 was 1:1) was respectively investigated using the CCK-8 assay. HaCaT cells and MNT-1 cells in the logarithmic phase were taken respectively, counted, diluted with a culture medium and plated in a 96-well plate. The cells were incubated at 5% CO2 and 37° C. After the cells were adhered to the wall, 100 μl of biomimetic liposome engineered with membrane proteins from keratinocytes (prepared in Example 10), and liposomes without membrane proteins (prepared in Example 8) with a concentration gradient of 0, 100, 500, and 1,000 μg/ml were added to the cells, respectively. After 24 h of incubation, 10 μl of CCK-8 solution was added to each well, and the incubation was continued for 2 h. The absorbance value of each well was measured at 450 nm of a microplate reader. Blank and control wells were set up, where the blank well was a culture medium containing CCK-8 but no cells or liposomes/biomimetic liposome engineered with membrane proteins from keratinocytes, and the control well was a culture medium containing cells and CCK-8 but no liposomes/biomimetic liposome engineered with membrane proteins from keratinocytes.

Cell viability %=[(Experimental well−Blank well)/(Control well−Blank well)]×100

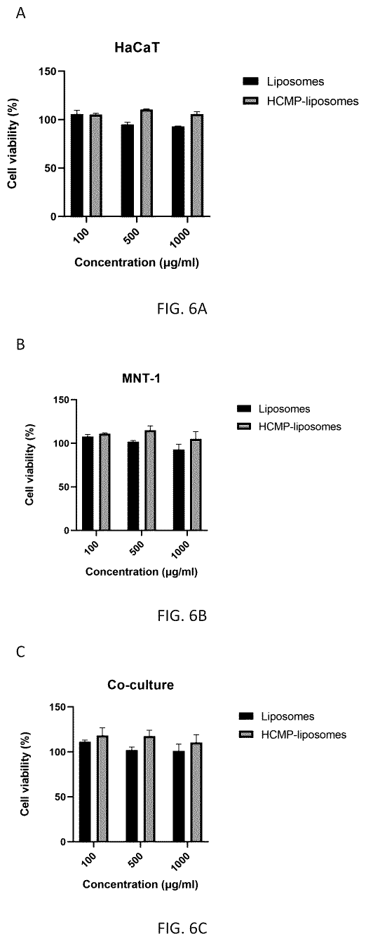

The results are shown in FIG. 6. It can be seen that compared with the untreated control well, the biomimetic liposome engineered with membrane proteins from keratinocytes and the liposomes without membrane proteins showed no significant cytotoxicity at all concentrations, and the biomimetic liposome engineered with membrane proteins from keratinocytes had higher proliferative effects on the HaCaT cells, the MNT-1 cells, and the co-culture model, which might be due to the fact that the biomimetic liposome engineered with membrane proteins from keratinocytes had better biocompatibility. 500 μg/ml of liposomes/biomimetic liposome engineered with membrane proteins from keratinocytes was selected as subsequent experiment concentration due to its highest proliferative effect on cells.

6. Investigation of Cellular Uptake of the Biomimetic Liposome Engineered with Membrane Proteins from Keratinocytes

To determine the differences in the uptake of liposomes and biomimetic liposome engineered with membrane proteins from keratinocytes by HaCaT cells and MNT-1 cells, the HaCaT cells and the MNT-1 cells were inoculated in a 6-well plate at a density of 5×10⁵ cells/well, respectively. After 24 h, the cells were washed twice with PBS at pH=7.4 and incubated with 1 mL of DMEM solution dispersed with 0.5 mg/ml rhodamine B-labeled biomimetic liposome engineered with membrane proteins from keratinocytes/liposomes (prepared in Examples 12 and 13), respectively, for 4 h at 37° C. The culture medium was removed, and the cells were washed three times with a fresh PBS at pH=7.4 and treated with 500 μL of 500 μg/mL trypsin in a calcium- and magnesium-free PBS for 2 min. DMEM (1 mL) supplemented with 10% FBS was added to the wells to quench the trypsin, and the cells were recovered and centrifuged at 1000 rpm for 5 min. Cell precipitates were resuspended in PBS at pH=7.4, washed twice with the same buffer, and recovered through centrifugation at 1000 rpm for 5 min. Cell samples were resuspended in 300 μL of PBS at pH=7.4 and analyzed by flow cytometry using a flow cytometer.

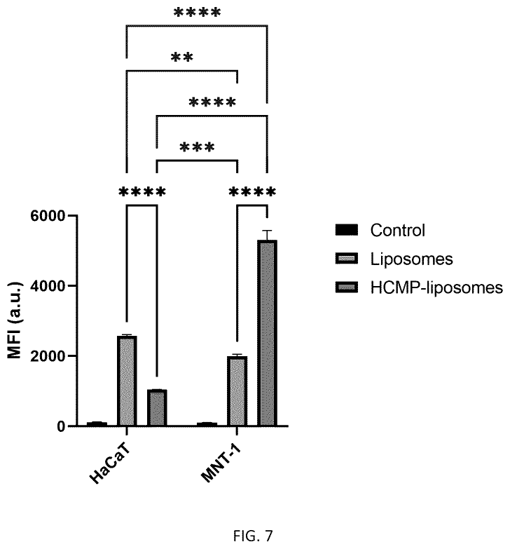

The results are shown in FIG. 7. It can be seen that compared with the liposomes, the uptake of the biomimetic liposome engineered with membrane proteins from keratinocytes by the HaCaT cells was significantly decreased, and the fluorescence intensity was decreased from 2578 to 1044, which was a 2.47-fold decrease; the uptake of the biomimetic liposome engineered with membrane proteins from keratinocytes by the MNT-1 cells was significantly increased, and the fluorescence intensity was increased from 1995.67 to 5301.33, which was a 2.66-fold increase; the uptake (fluorescence intensity) of the biomimetic liposome engineered with membrane proteins from keratinocytes by the MNT-1 cells was 5.1-fold higher than that of the HaCaT cells, which proved that the prepared biomimetic liposome engineered with membrane proteins from keratinocytes were significantly targeted to melanocytes.

7. Results of Flow Cytometry Analysis of the Biomimetic Liposome Engineered with Membrane Proteins from Keratinocytes for Inhibition of Melanosome Transport in a Co-Culture Model.

To quantitatively measure melanosome transport, the HaCaT cells and the MNT-1 cells were inoculated at a ratio of 2:1 in a six-well plate at a density of 5×10⁵ cells/well. The culture medium was consisted of a HaCaT culture medium and an MNT-1 culture medium at a ratio of 2:1. After 24 hours of cultivation, the cells were washed twice with PBS at pH=7.4 and incubated with 1 mL of 500 μg/ml biomimetic liposome engineered with membrane proteins from keratinocytes (prepared in Example 10) for 24 h at 37° C. The co-cultured cells were harvested, washed with cold PBS, fixed in 4% paraformaldehyde for 10 min, and washed with PBS containing 0.1% Triton-X100 for 5 min. The MNT-1 cells were immunostained with PMEL 17 rabbit monoclonal antibody (Beyotime, AG8635) and Alexa Fluor 488-labeled goat anti-rabbit IgG (H+L) (Beyotime, A0423), and the HaCaT cells were incubated with anti-pan-cytokeratin mouse recombinant polyclonal antibody (abcam, ab86734) and Alexe Fluor 647-labeled goat anti-mouse IgG (H+L) (Beyotime, A0473). Stained cells were analyzed by flow cytometry, and a total of 10000 cells were collected on a flow cytometer.

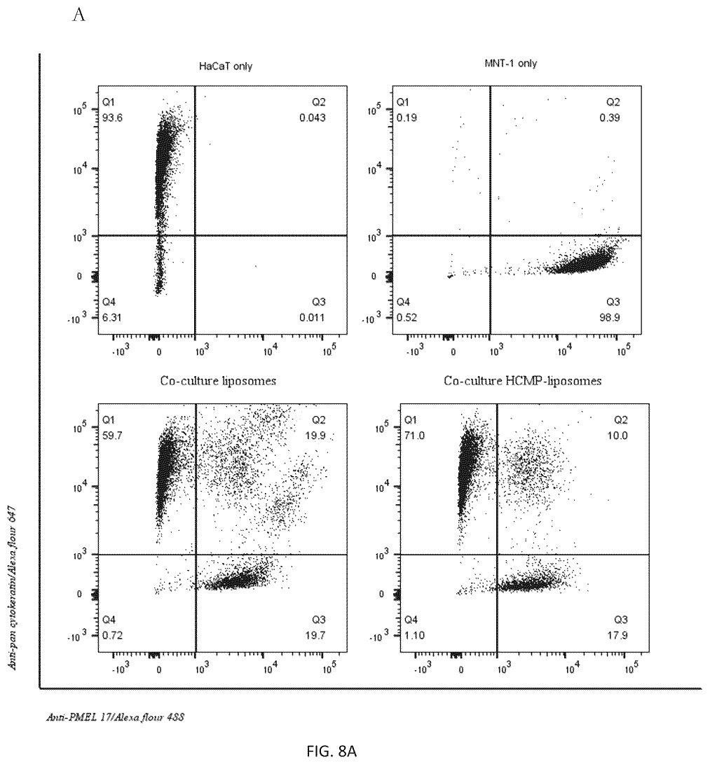

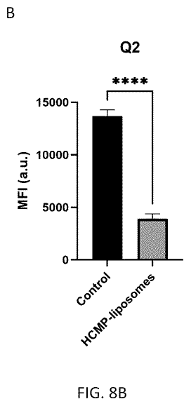

The results are shown in FIG. 8. It can be seen that compared with the untreated control well, the fluorescence intensity of Alexa Fluor 488 representing melanosome in the keratinocytes was significantly decreased from 13649 to 3917, which was a 3.5-fold decrease, in the co-culture cell model treated with the biomimetic liposome engineered with membrane proteins from keratinocytes, indicating that the biomimetic liposome engineered with membrane proteins from keratinocytes can inhibit the melanin transport process to skin cells.

8. Result of Laser Confocal Analysis of the Biomimetic Liposome Engineered with Membrane Proteins from Keratinocytes for Inhibition of Melanosome Transport in a Co-Culture Model.

To quantitatively measure melanosome transport, the HaCaT cells and the MNT-1 cells were inoculated at a ratio of 2:1 in a six-well plate at a density of 5×10⁵ cells/well. The culture medium was consisted of a HaCaT culture medium and an MNT-1 culture medium at a ratio of 2:1. After 24 hours of cultivation, the cells were washed twice with PBS at pH=7.4 and incubated with 1 mL of 500 μg/ml biomimetic liposome engineered with membrane proteins from keratinocytes (prepared in Example 10) for 24 h at 37° C. The co-cultured cells were harvested, washed with cold PBS, fixed in 4% paraformaldehyde for 10 min, and washed with PBS containing 0.1% Triton-X100 for 5 min. The MNT-1 cells were immunostained with PMEL 17 rabbit monoclonal antibody (Beyotime, AG8635) and Alexa Fluor 488-labeled goat anti-rabbit IgG (H+L) (Beyotime, A0423), and the HaCaT cells were incubated with anti-pan-cytokeratin mouse recombinant polyclonal antibody (abcam, ab86734) and Alexe Fluor 647-labeled goat anti-mouse IgG (H+L) (Beyotime, A0473). The number of melanosomes within the keratinocytes was analyzed by a laser confocal microscope.

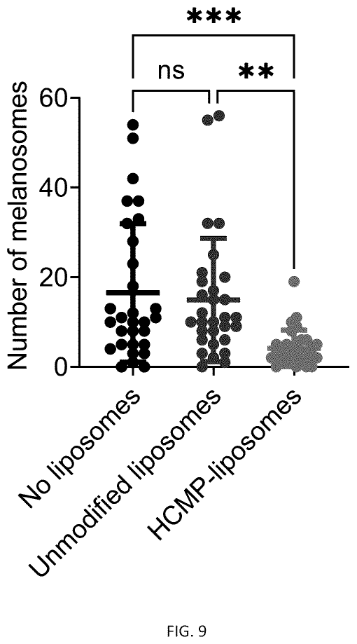

The results are shown in FIG. 9. It can be seen that compared with the untreated control well, the number of fluorescent bright spots of Alexa Fluor 488 representing melanosome in the keratinocytes was significantly decreased from an average of 16.5 per cell to 4.1 per cell, which was a 4.02-fold decrease, in the co-culture cell model treated with the biomimetic liposome engineered with membrane proteins from keratinocytes, which was consistent with the results of flow cytometry analysis, indicating that the biomimetic liposome engineered with membrane proteins from keratinocytes can inhibit the melanin transport process to skin cells.

### Comparative Example 1: Effect of Total Lipid Concentration on Biomimetic Liposome Engineered with Membrane Proteins from Keratinocytes

The effect of total lipid concentration of 5 and 20 mg/ml on the preparation of biomimetic liposome engineered with membrane proteins from keratinocytes was investigated.

A mixture of soy lecithin and cholesterol in a molar ratio of 7:3 was dissolved in anhydrous ethanol at a total lipid concentration of 5 mg/ml and prepared as an organic phase. 1×PBS buffer without the addition of keratinocyte membrane protein was used as an aqueous phase. The aqueous phase was used as an external phase and the organic phase was used as an internal phase. A microfluidic chip (Wuhan Jianmizhikong Technology Co., Ltd.) was focused via fluid at a total flow rate (TFR) of 1 ml/min and a flow rate ratio (aqueous phase/organic phase=FRR) of 6:1. An obtained solution was dialyzed overnight with a 300 kDa dialysis bag to obtain the liposome. As the lipid concentration was too low, the particle size was as small as 57.6 nm and the polydispersity index was as large as 0.271 for the obtained liposome.

A mixture of soy lecithin and cholesterol in a molar ratio of 7:3 was dissolved in anhydrous ethanol at a total lipid concentration of 20 mg/ml and prepared as the organic phase. 1×PBS buffer without the addition of keratinocyte membrane protein was used as the aqueous phase. The aqueous phase was used as the external phase and the organic phase was used as the internal phase. A microfluidic chip (Wuhan Jianmizhikong Technology Co., Ltd.) was focused via fluid at a total flow rate (TFR) of 1 ml/min and a flow rate ratio (aqueous phase/organic phase=FRR) of 6:1. An obtained solution was dialyzed overnight with a 300 kDa dialysis bag to obtain the liposome. As the lipid concentration was too high, the particle size was as large as 207.4 nm, the polydispersity index was increased dramatically to 0.352 and aggregation was observed for the obtained liposome.

### Comparative Example 2: Effect of FRR on Biomimetic Liposome Engineered with Membrane Proteins from Keratinocytes

A mixture of soy lecithin and cholesterol in a molar ratio of 7:3 was dissolved in anhydrous ethanol at a total lipid concentration of 10 mg/ml and prepared as an organic phase. 1×PBS buffer without the addition of keratinocyte membrane protein was used as an aqueous phase. The aqueous phase was used as an external phase and the organic phase was used as an internal phase. A microfluidic chip (Wuhan Jianmizhikong Technology Co., Ltd.) was focused via fluid at a total flow rate (TFR) of 1 ml/min and a flow rate ratio (aqueous phase/organic phase=FRR) of 8:1. An obtained solution was dialyzed overnight with a 300 kDa dialysis bag to obtain the liposome. As the flow rate ratio was too large, laminar diffusion could not be formed in the chip channel, making it difficult to prepare the liposome.

### Comparative Example 3: Effect of TFR on Biomimetic Liposome Engineered with Membrane Proteins from Keratinocytes

A mixture of soy lecithin and cholesterol in a molar ratio of 7:3 was dissolved in anhydrous ethanol at a total lipid concentration of 10 mg/ml and prepared as an organic phase. 1×PBS buffer without the addition of keratinocyte membrane protein was used as an aqueous phase. The aqueous phase was used as an external phase and the organic phase was used as an internal phase. A microfluidic chip (Wuhan Jianmizhikong Technology Co., Ltd.) was focused via fluid at a total flow rate (TFR) of 2 ml/min and a flow rate ratio (aqueous phase/organic phase=FRR) of 6:1. An obtained solution was dialyzed overnight with a 300 kDa dialysis bag to obtain the liposome. As the total flow rate was too high, the chip could not withstand the pressure and resulted to leakage.

### Comparative Example 4: Effect of Lipid Species on Biomimetic Liposome Engineered with Membrane Proteins from Keratinocytes

The effect of adding dioleoyl phosphatidylethanolamine and dioleoyl phosphatidylcholine into lipid species on the preparation of biomimetic liposome engineered with membrane proteins from keratinocytes was investigated.

A mixture of soy lecithin, cholesterol and dioleoyl phosphatidylethanolamine (DOPE) in a molar ratio of 7:3:1 was dissolved in anhydrous ethanol at a total lipid concentration of 10 mg/ml and prepared as an organic phase. A 1×PBS buffer added with keratinocyte membrane protein at a concentration of 0.05 mg/ml was used as an aqueous phase. The aqueous phase was used as an external phase and the organic phase was used as an internal phase. A microfluidic chip (Wuhan Jianmizhikong Technology Co., Ltd.) was focused via fluid at a total flow rate (TFR) of 1 ml/min and a flow rate ratio (aqueous phase/organic phase=FRR) of 6:1. An obtained solution was dialyzed overnight with a 300 kDa dialysis bag to obtain the biomimetic liposome engineered with membrane proteins from keratinocytes. Since DOPE was a cationic lipid, its binding to negatively charged groups in membrane proteins during self-assembly in diffusion leads to a limited self-assembly process, resulting in aggregation of the biomimetic liposome engineered with membrane proteins from keratinocytes with a polydispersity index of 0.41.

A mixture of soy lecithin, cholesterol and dioleoyl phosphatidylcholine (DOPC) in a molar ratio of 7:3:1 was dissolved in anhydrous ethanol at a total lipid concentration of 10 mg/ml and prepared as the organic phase. A 1×PBS buffer added with keratinocyte membrane protein at a concentration of 0.05 mg/ml was used as the aqueous phase. The aqueous phase was used as an external phase and the organic phase was used as an internal phase. A microfluidic chip (Wuhan Jianmizhikong Technology Co., Ltd.) was focused via fluid at a total flow rate (TFR) of 1 ml/min and a flow rate ratio (aqueous phase/organic phase=FRR) of 6:1. An obtained solution was dialyzed overnight with a 300 kDa dialysis bag to obtain the biomimetic liposome engineered with membrane proteins from keratinocytes. The obtained biomimetic liposome engineered with membrane proteins from keratinocytes had a relatively high Zeta potential of −16.9 mV, which was unfavorable for the embedding of membrane proteins into the phospholipid bilayer.

### Comparative Example 5: Effect of Cell Membrane Protein Concentration on Biomimetic Liposome Engineered with Membrane Proteins from Keratinocytes

The effect of cell membrane protein concentrations of 0.2 mg/ml and 0.01 mg/ml on the preparation of biomimetic liposome engineered with membrane proteins from keratinocytes was investigated.

A mixture of soy lecithin and cholesterol in a molar ratio of 7:3 was dissolved in anhydrous ethanol at a total lipid concentration of 10 mg/ml and prepared as an organic phase. A 1×PBS buffer added with keratinocyte membrane protein at a concentration of 0.2 mg/ml was used as an aqueous phase. The aqueous phase was used as an external phase and the organic phase was used as an internal phase. A microfluidic chip (Wuhan Jianmizhikong Technology Co., Ltd.) was focused via fluid at a total flow rate (TFR) of 1 ml/min and a flow rate ratio (aqueous phase/organic phase=FRR) of 6:1. An obtained solution was dialyzed overnight with a 300 kDa dialysis bag to obtain the biomimetic liposome engineered with membrane proteins from keratinocytes. As the membrane protein concentration was too high, the self-assembly process of phospholipids in diffusion was limited, resulting in a heterogeneous particle size distribution of the obtained biomimetic liposome engineered with membrane proteins from keratinocytes with a polydispersity index of 0.37.

A mixture of soy lecithin and cholesterol in a molar ratio of 7:3 was dissolved in anhydrous ethanol at a total lipid concentration of 10 mg/ml and prepared as the organic phase. A 1×PBS buffer added with keratinocyte membrane protein at a concentration of 0.01 mg/ml was used as the aqueous phase. The aqueous phase was used as an external phase and the organic phase was used as an internal phase. A microfluidic chip (Wuhan Jianmizhikong Technology Co., Ltd.) was focused via fluid at a total flow rate (TFR) of 1 ml/min and a flow rate ratio (aqueous phase/organic phase=FRR) of 6:1. An obtained solution was dialyzed overnight with a 300 kDa dialysis bag to obtain the biomimetic liposome engineered with membrane proteins from keratinocytes. As the cell membrane protein concentration was too low, the Zeta potential of the prepared biomimetic liposome engineered with membrane proteins from keratinocytes differed very little from that of the liposome without membrane proteins, and the protein density on the surface of the liposome was too low and the bioactivity was poor.

### Comparative Example 6: Effect of Presence of Cell Membrane Protein on Cellular Uptake

A mixture of soy lecithin and cholesterol in a molar ratio of 7:3 was dissolved in anhydrous ethanol at a total lipid concentration of 10 mg/ml, while rhodamine B-DHPE at a concentration of 0.2 mol % was added, and prepared as an organic phase. 1×PBS buffer containing only the same concentration of membrane protein extraction reagent B (without membrane protein) was added and used as an aqueous phase. The aqueous phase was used as an external phase and the organic phase was used as an internal phase. A microfluidic chip (Wuhan Jianmizhikong Technology Co., Ltd.) was focused via fluid at a total flow rate (TFR) of 1 ml/min and a flow rate ratio (aqueous phase/organic phase=FRR) of 6:1. An obtained solution was dialyzed overnight with a 300 kDa dialysis bag to obtain the liposome. The prepared liposomes were incubated with HaCaT cells and MNT-1 cells, respectively. Due to the absence of cell membrane proteins, the prepared liposomes showed no significant change in cellular uptake compared to control liposomes (prepared in Example 13, in which the aqueous phase was PBS buffer without membrane proteins), and the effect of the biomimetic liposome engineered with membrane proteins from keratinocytes on cellular uptake was due to the presence of surface membrane proteins.

### Comparative Example 7: Effect of Presence of Cell Membrane Protein on the Inhibition of Melanosome Transport

A mixture of soy lecithin and cholesterol in a molar ratio of 7:3 was dissolved in anhydrous ethanol at a total lipid concentration of 10 mg/ml, while rhodamine B-DHPE at a concentration of 0.2 mol % was added, and prepared as an organic phase. 1×PBS buffer containing only the same concentration of membrane protein extraction reagent B (without membrane protein) was added and used as an aqueous phase. The aqueous phase was used as an external phase and the organic phase was used as an internal phase. A microfluidic chip (Wuhan Jianmizhikong Technology Co., Ltd.) was focused via fluid at a total flow rate (TFR) of 1 ml/min and a flow rate ratio (aqueous phase/organic phase=FRR) of 6:1. An obtained solution was dialyzed overnight with a 300 kDa dialysis bag to obtain the liposome. After incubating the prepared liposomes with a co-culture model, the transport efficiency of melanosome was detected through flow cytometry. Due to the absence of cell membrane proteins, the prepared liposomes and control liposomes (prepared in Example 13) had no effect on inhibiting melanosome transport, and there was no significant change in the fluorescence intensity of melanosomes within the keratinocytes as compared to a control co-culture model group.

Although the present disclosure has been disclosed above by preferred examples, the present disclosure is not limited thereto. Any person familiar with the technology can make various changes and modifications without departing from the spirit and scope of the present disclosure, and therefore the protection scope of the present disclosure should be subject to the scope defined by the claims.

What is claimed is:

1. A biomimetic liposome, wherein the biomimetic liposome is a nanoscale phospholipid bilayer vesicle with an outer membrane consisting of a phospholipid bilayer and membrane proteins from cells and exosomes.

2. The biomimetic liposome according to claim 1, wherein the cell membrane proteins comprises keratinocyte membrane proteins but also include other membrane proteins macrophages, cancer cells, etc. and their exosomes.

3. An example of biomimetic liposome according to claim 1, wherein the mass ratio of the keratinocyte membrane proteins to the phospholipid bilayer is 1:(50-500).

4. The biomimetic liposome according to claim 2, wherein the phospholipid bilayer comprises lecithin and cholesterol, and the molar ratio of the lecithin to the cholesterol is (3-2):1.

5. The biomimetic liposome according to claim 3, wherein the keratinocyte membrane proteins are membrane proteins extracted from keratinocytes.

6. The biomimetic liposome according to claim 4, wherein the keratinocytes comprise human immortalized keratinocytes.

7. A method for preparing the biomimetic liposome according to claim 1, wherein the method comprises the following steps:
(1) preparation of organic phase: mixing and dissolving lecithin and cholesterol to obtain the organic phase;
(2) preparation of aqueous phase: dissolving keratinocyte membrane proteins to obtain the aqueous phase; and
(3) using the aqueous phase obtained in step (2) as an external phase, using the organic phase obtained in step (1) as an internal phase, and focusing a microfluidic chip via fluid to obtain the biomimetic liposome.

8. The method according to claim 6, wherein the total flow rate TFR at which the microfluidic chip is focused via fluid in step (3) is 0.1 to 1 ml/min.

9. The method according to claim 7, wherein the flow rate ratio FRR of aqueous phase/organic phase at which the microfluidic chip is focused via fluid in step (3) is (3-6):1.

10. The method according to claim 8, wherein after the microfluidic chip is focused via fluid in step (3), an obtained solution is collected for dialysis; and the molecular weight cut-off for dialysis is 30 to 300 kDa.

11. The method according to claim 9, wherein the molar ratio of the lecithin to the cholesterol in the organic phase in step (1) is (3-2):1.

12. The method according to claim 9, wherein the concentration of the organic phase in step (1) is 5 to 10 mg/ml.

13. The method according to claim 11, wherein the concentration of the aqueous phase in step (2) is 0.033 to 0.1 mg/ml.

14. A method for inhibiting melanin transfer in skin using the biomimetic liposome according to claim 1, wherein the biomimetic liposome or a product comprising the biomimetic liposome is used as a cosmetic, a skincare product, or a medicine, the medicine comprising a topically applied medicine or an orally administered medicine.

15. A method for treating skin hyperpigmentation using the biomimetic liposome according to claim 1, wherein the biomimetic liposome or a product comprising the biomimetic liposome is used as a cosmetic, a skincare product, or a medicine, the medicine comprising a topically applied medicine or an orally administered medicine; and the skin hyperpigmentation comprises colour spots, chloasma, cyasma, sunspots, or pigmentation after wound healing.
